(19) United States
(12) Patent Application Publication
(10) Pub. No.: US 2024/0156600 A1
(43) Pub. Date: May 16, 2024

(54) BALLOON EPICARDIAL ANCHOR

(71) Applicant: EDWARDS LIFESCIENCES CORPORATION, Irvine, CA (US)

(72) Inventors: Jason Scott Provol, San Diego, CA (US); Arvin T. Chang, Yorba Linda, CA (US)

(21) Appl. No.: 18/423,147

(22) Filed: Jan. 25, 2024

Related U.S. Application Data
(63) Continuation of application No. PCT/US2022/037754, filed on Jul. 20, 2022.
(60) Provisional application No. 63/226,704, filed on Jul. 28, 2021.

(51) Int. Cl. A61F 2/24; A61B 17/34

(52) U.S. Cl. CPC A61F 2/2487; A61B 17/3468; A61F 2210/0085; A61F 2220/0008; A61F 2250/0003

(57) ABSTRACT

A method and device for placing an epicardial anchor in a heart are described. An anchor placement device includes a tube, a needle formed on a distal end of the tube, an inflatable balloon in proximity to the distal end of the tube, and a control plate located proximally from the inflatable balloon. The anchor placement device is configured to pierce from an endocardium layer of the heart through an epicardium layer of the heart and then inflate the balloon to form an anchor located outside the epicardium layer. Meanwhile, the control plate is configured to abut against the endocardium layer and reduce movement of the tube while the balloon is inflated.

### RELATED APPLICATION

This application is a continuation of International Patent Application No. PCT/US2022/037754, filed Jul. 20, 2022 and entitled BALLOON EPICARDIAL ANCHOR, which claims priority to U.S. Provisional Patent Application No. 63/226,704, filed Jul. 28, 2021 and entitled BALLOON EPICARDIAL ANCHOR, the complete disclosures of which are hereby incorporated by reference in their entireties.

### BACKGROUND

### Field

The present disclosure generally relates to the field of heart implant devices and implant techniques.

### Description of Related Art

Heart failure can occur when the left ventricle of the heart becomes enlarged and dilated due to various conditions, such as chronic hypertension, myocardial infarction, mitral valve incompetency, and other dilated cardiomyopathies. Treatments for such heart dysfunctions can involve implanting devices in the heart, either temporarily or permanently. Such devices may need to be anchored in the heart to keep the devices in place or to transfer force to the heart walls.

### SUMMARY

Described herein are one or more methods and/or devices to deploy an anchor that lies outside the epicardium, the anchor usable in medical procedures or for anchoring implants.

One general aspect includes a device for placing an anchor in a heart. The device includes a tube and a needle formed on a distal end of the tube, the needle configured to pierce from an endocardium layer of the heart through an epicardium layer of the heart. The device can include an inflatable balloon in proximity to the distal end of the tube, the inflatable balloon configured to inflate and form an anchor located outside the epicardium layer. The device can also include a control plate located proximally from the inflatable balloon, the control plate configured to abut against the endocardium layer and reduce movement of the tube while the inflatable balloon is inflated.

Implementations may include one or more of the following features. The device may include: a conduit in the tube for releasing curable adhesive into the inflatable balloon, the curable adhesive configured to inflate the balloon. The device may include: a fiber optic cable configured to emit ultraviolet (UV) light, the UV light configured to cure the adhesive in the inflatable balloon and permanently leave the inflatable balloon in its inflated configuration. The fiber optic cable may run longitudinally along a surface of the tube. The fiber optic cable and the conduit may be in separate lumens in the tube. The control plate may be attached to an external surface of the catheter, the control plate configured to hold the catheter steady while the tube and inflatable balloon are moved to a target location. Alternatively, the control plate can be attached to the tube at a fixed distance from the needle, the control plate configured to prevent the needle from passing further than the fixed distance from the endocardium layer. The inflatable balloon may be configured to form a generally disk shape, a generally cylindrical shape, or a generally saucer shape. The device may include a suture attached to the inflatable balloon. The inflatable balloon may be further configured to detach from the tube.

One general aspect includes a method for deploying an anchor to an epicardium layer of a heart. The method includes advancing a tube to an interior chamber of the heart. The tube may include a needle formed on a distal end of the tube, an inflatable balloon in proximity to the distal end of the tube, and a control plate located proximally from the inflatable balloon. The method can include anchoring the control plate to an interior surface of an endocardium layer of the heart. The method can include advancing the needle from the endocardium layer of the heart through an epicardium layer of the heart. The method can also include deploying the inflatable balloon to form an anchor against an exterior surface of the epicardium layer.

Implementations may include one or more of the following features. The method for deploying the inflatable balloon may include: releasing a curable adhesive into the inflatable balloon to expand the inflatable balloon to a target size, and curing the adhesive within the inflatable balloon to permanently set the inflatable balloon into an anchor configuration. Curing the adhesive may include: deploying a UV emitting cable into the inflatable balloon to apply UV light to the curable adhesive, and setting the adhesive into a rigid material that forms the inflatable balloon into the anchor configuration. The method may include: releasing the inflatable balloon from the tube, leaving the inflatable balloon anchored to the epicardium layer, the inflatable balloon attached to a suture, and withdrawing the tube from the interior chamber of the heart. The method may include: advancing a catheter containing the tube to the interior chamber of the heart, the control plate attached to an exterior surface of the catheter. The method may include: releasing the inflatable balloon from the tube, leaving the inflatable balloon as a first anchor at the epicardium layer, the inflatable balloon attached to a suture, withdrawing the tube from the catheter, moving the catheter to a second location, advancing a second tube having a second inflatable balloon into the catheter, and deploying the second inflatable balloon as a second anchor at the second location. The method may include: deploying a cinching device connected, via a suture, to the first anchor and the second anchor, and cinching the suture to reduce a size of a structure of the heart, the structure located between the first anchor and the second anchor.

One general aspect includes a needle for placing an anchor in a heart. The needle can include an inflatable balloon in proximity to a distal end of the needle, the inflatable balloon configured to: inflate and form an anchor located outside the epicardium layer, and detach from the needle. The needle can also include a suture attached to the inflatable balloon. The needle can also include a control plate located proximally from the inflatable balloon, the control plate configured to abut against the endocardium layer and reduce movement of the needle while the inflatable balloon is inflated.

### BRIEF DESCRIPTION OF THE DRAWINGS

Various examples are depicted in the accompanying drawings for illustrative purposes, and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1A provides a cross-sectional view of a human heart.

FIG. 1B illustrates a cross-sectional view of a heart wall.

FIG. 2 illustrates one possible medical procedure where an anchor deployment device can be used, according to certain examples.

FIGS. 3A and 3B illustrate an example of the anchor placement device.

FIGS. 4A and 4B illustrate another example of the anchor placement device.

FIGS. 5A-5C illustrate various examples of an inflated balloon of the anchor deployment device.

FIGS. 6A-6D illustrate a deployment of the inflatable balloon into its anchor configuration, according to certain examples.

FIGS. 7A and 7B illustrate a side view of the anchor placement device as the inflated balloon is detached, according to certain examples.

FIG. 8 illustrates a tube of the anchor placement device having an optical cable built into its structure, according to certain examples.

FIG. 9 provides a flow diagram representing a process for using the anchor placement device to perform a medical procedure, according to certain examples.

### DETAILED DESCRIPTION

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, with respect to any method or process disclosed herein, the acts or operations of the method or process may be performed in any suitable sequence and are not necessarily limited to any particular disclosed sequence.

Various operations may be described as multiple discrete operations in turn, in a manner that may be helpful in understanding certain examples; however, the order of description should not be construed to imply that these operations are order dependent. Additionally, the structures, systems, and/or apparatuses/devices described herein may be embodied as integrated components or as separate components. For purposes of comparing various examples, certain aspects and advantages of these examples are described. Furthermore, the headings provided herein are for convenience only and do not necessarily affect the scope or meaning of the claimed invention.

Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein Similar reference numbers may be used with respect to separate diagrams and/or examples; use of such similar, or identical, reference numbers should not be interpreted as necessarily identifying identical components, and may refer to separate features.

### Overview

Heart failure can occur when the left ventricle of the heart becomes enlarged and dilated due to various conditions, such as chronic hypertension, myocardial infarction, mitral valve incompetency, and other dilated cardiomyopathies. With each of these conditions, the heart is forced to overexert itself in order to provide a cardiac output demanded by the body during various demand states. The result can be an enlarged left ventricle. A dilated or enlarged heart, and particularly a dilated or enlarged left ventricle, can significantly increase tension and stress in heart walls both during diastolic filling and systolic contraction, which contributes to further dilatation or enlargement of chambers of the heart.

One potential type of treatment involves reshaping the heart. A normal heart is elliptical and shaped like a football, while the dilated heart is more spherical in shape, like a basketball. Reshaping the heart can alter its size and shape to make the dilated basketball-shaped heart smaller and restore the more normal football shape. One surgical procedure involves applying one or more splints onto the heart, to reduce myocardial muscular stresses encountered during pumping. Each splint may include a tension member or suture extending across the ventricle with anchors disposed on opposite ends of the tension member and placed on the external surface of the heart.

The surface area of an anchor and/or size of the anchor can correspond to the ability of an anchor to withstand forces due to tension from reshaping the heart and ongoing beating of the heart (although, other design features and material properties may also contribute to the ability of the anchor to withstand tension forces). For greater effectiveness and safety, anchors would ideally be able to withstand high forces, including forces as high as 17 Newtons (N) or higher, while the splint maintains the heart in a desired shape. Further, the anchor should have a large enough surface area to spread out and reduce the pressure on the myocardium. If the pressure gets too high on an area (e.g., a small, focused pressure area) of the heart, this can lead to myocardium necrosis, which can itself lead to migration and sinking of the anchor into the tissue. Accordingly, large anchors, or anchors with a large surface area, may be required, and the larger size/area can make implantation of the anchor difficult and can require opening the heart, chest, and/or sternum, and/or may require other highly invasive procedures.

Currently available methods of mitral valve repair or replacement typically require opening the chest and/or heart, e.g., to gain direct access to the valve and its annulus or another portion of the heart. This type of access typically necessitates a use of cardiopulmonary bypass, which can introduce additional complications to the surgical procedure. Since the implantation of the splints themselves do not require the patient to be on cardiopulmonary bypass, it would be advantageous to devise a technique which could improve the mitral valve without any need for cardiopulmonary bypass. The ability to improve the mitral valve function without the need for cardiopulmonary bypass would be an advantage, both in conjunction with ventricular splinting, and also as a stand-alone therapy. Indeed, it would be desirable to have systems, apparatuses, and methods capable of a deploying an anchor with an ability to withstand high pressures (e.g., an anchor having a large surface area) using a less invasive, or minimally invasive procedure.

The following disclosure discuses an anchor deployment device for deploying anchors to the outside of the heart from within the heart. By placing the anchor outside the heart, external to the epicardial layer, the anchor is able to resist stronger forces. In certain examples, one or more sutures are connected to the anchor and attached to other medical devices, such as other anchors, a cinching mechanism, an implant, and/or the like. However, in order to reach the epicardium, the anchor has to puncture through the myocardium layer, which may have undesirable consequences, such as causing a high chance of effusion. Thus, certain examples utilize a small needle and an expandable anchor that can pierce through the myocardium while leaving relatively little damage. Once the anchor passes through the myocardium and past the epicardium, the anchor can be expanded to create a larger surface area to help the anchor withstand higher forces.

### Implantation Location

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

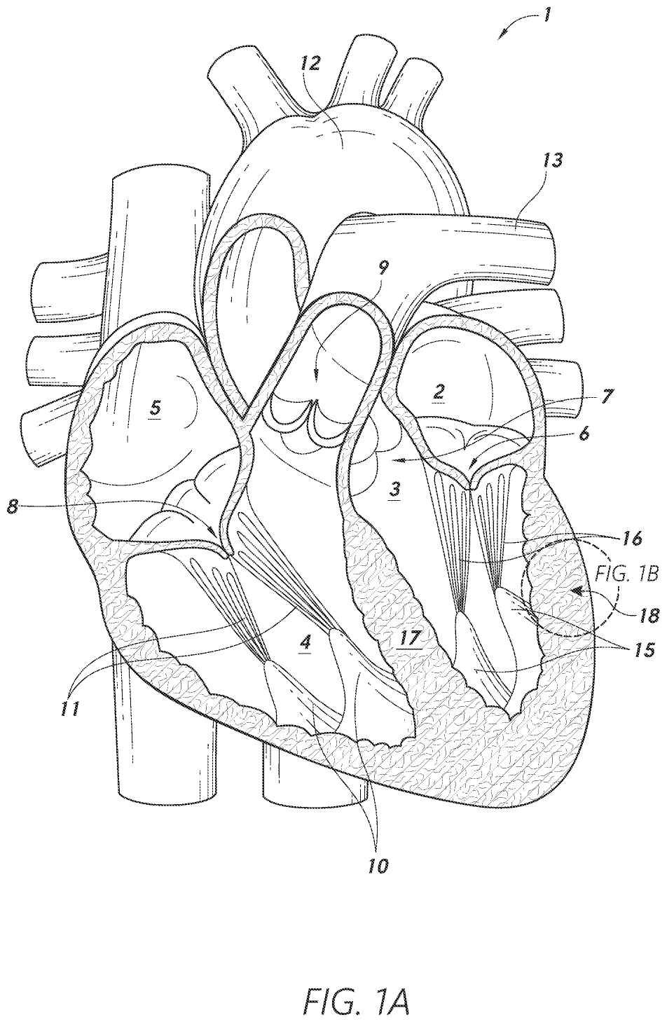

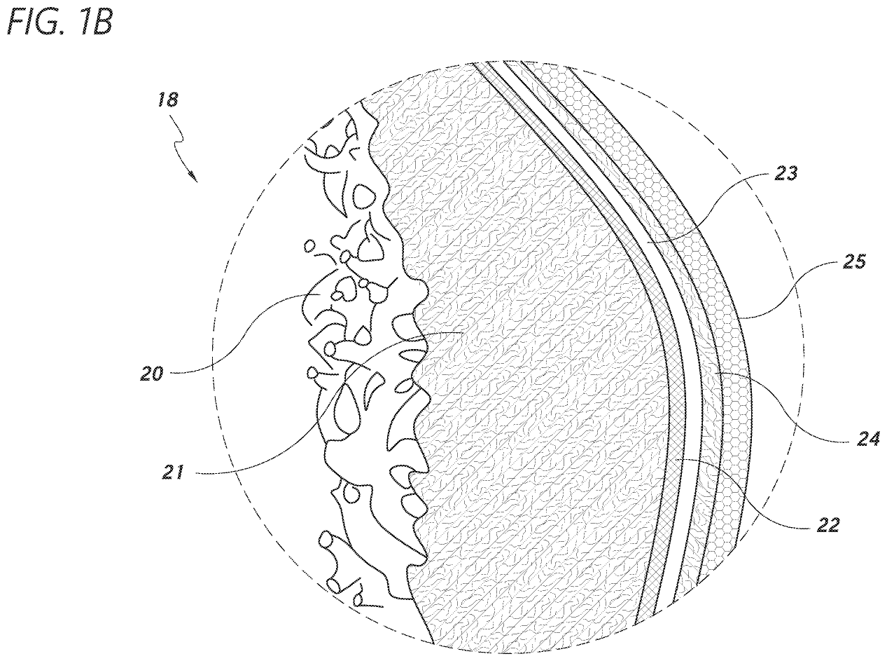

FIG. 1A illustrates an example representation of a heart 1 having various features relevant to certain examples of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 13, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other such that the leaflets/cusps coapt, thereby closing the flow passage.

The atrioventricular (i.e., mitral 6 and tricuspid 8) heart valves may further comprise a respective collection of chordae tendineae (16, 11) and papillary muscles (15, 10) for securing the leaflets of the respective valves to promote and/or facilitate proper coaptation of the valve leaflets and prevent prolapse thereof. The papillary muscles (15, 10) may generally comprise finger-like projections from the ventricle wall, while the chordae tendineae (16, 11) may comprise cord-like tendons that connect the papillary muscles to the valve leaflets.

With respect to the mitral valve 6, a normal mitral valve may comprise two leaflets (anterior and posterior) and chordae tendineae 16 connecting the leaflets to two corresponding papillary muscles 15. The papillary muscles 15 originate in the left ventricle wall and project into the left ventricle 3. The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure can force the valve to close, while the chordae tendineae 16 may keep the leaflets coapting together and prevent the valve from opening in the wrong direction, thereby preventing blood to flow back to the left atrium 2. The various cords of the chordae tendineae may have different thicknesses, wherein relatively thinner cords are attached to the free leaflet margin, while relatively thicker cords (e.g., strut cords) are attached farther away from the free margin.

With respect to the tricuspid valve 8, a normal tricuspid valve may comprise three leaflets (two shown in FIG. 1A) and three corresponding papillary muscles 10 (two shown in FIG. 1A). The leaflets of the tricuspid valve 8 may be referred to as the anterior, posterior and septal leaflets, respectively. The valve leaflets are connected to the papillary muscles by the chordae tendineae 11, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle binding and/or adjustment disclosed herein are applicable to atrioventricular valves having any number of leaflets and/or chordae tendineae or papillary muscles associated therewith. The right ventricular papillary muscles 10 originate in the right ventricle wall, and attach to the anterior, posterior and septal leaflets of the tricuspid valve, respectively, via the chordae tendineae 11. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is in a closed state. In certain conditions, valve disease can be severely debilitating and even fatal if left insufficiently treated. With regard to incompetent heart valves, over time and/or due to various physiological conditions, the position and/or tension of the chordae tendineae and/or papillary muscles may become altered, thereby pulling the valve leaflets at least partly open, which may cause valve regurgitation. For example, functional mitral valve regurgitation can occur when the left ventricle of the heart is distorted or dilated, displacing the papillary muscles, and chordae tendineae attached thereto, that support the mitral valve leaflets. For example, the valve leaflets may no longer come together to close the annulus, thereby resulting in blood flow back into the atrium. If left untreated, functional mitral valve regurgitation can overload the heart and can lead to or accelerate heart failure. Moving or pulling the chordae tendineae closer to the flow axis of the valve annulus according to their natural and healthy positions can potentially reduce occurrence of valve regurgitation.

FIG. 1B illustrates a cross section of the heart wall tissue, such as tissue from the heart wall 18 in FIG. 1A. As shown in FIG. 1B, the heart walls 18 comprise several layers, including, from the inner layer to the outer layer, the endocardium 20, the myocardium 21, and the epicardium 22. The pericardium 24, 25 surrounds the heart, with a fluid filled space between called the pericardial cavity 23 lying between the pericardium and the epicardium.

The endocardium 20 is the thin inner layer of the heart. This layer lines the inner heart chambers, covers heart valves, and is continuous with the endothelium of large blood vessels. The endocardium of heart atria consists of smooth muscle, as well as elastic fibers. The myocardium 21 is the muscular middle layer wall of the heart. It is composed of cardiac muscle fibers, which enable heart contractions. The myocardium is the thickest layer of the heart wall, with its thickness varying in different parts of the heart.

The epicardium 22 is the outer protective layer of the heart. It is also known as visceral pericardium as it forms the inner layer of the pericardium. The epicardium is composed primarily of loose connective tissue, including elastic fibers and adipose tissue. The epicardium functions to protect the inner heart layers and also assists in the production of pericardial fluid. This fluid fills the pericardial cavity and helps to reduce friction between pericardial membranes. Also found in this heart layer are the coronary blood vessels, which supply the heart wall with blood. The inner layer of the epicardium is in direct contact with the myocardium.

The pericardium 24, 25, also called pericardial sac, is a double-walled sac containing the heart. It covers the heart from all sides except at the cardiac root at the bottom, where the great vessels join the heart. It has two layers, an outer layer made of strong connective tissue (fibrous pericardium 25), and an inner layer made of serous membrane (serous pericardium 24). It encloses the pericardial cavity 23, which contains pericardial fluid, and defines the middle mediastinum. It separates the heart from interference of other structures, protects it against infection and blunt trauma, and lubricates the heart's movements. The same mesothelium 22, 24 that constitutes the serous pericardium 24 also covers the heart as the epicardium 22, resulting in a continuous serous membrane invaginated onto itself as two opposing surfaces (over the fibrous pericardium 25 and over the heart). This creates a pouch-like potential space around the heart enclosed between the two opposing serosal surfaces, known as the pericardial space or pericardial cavity 23, which is filled with a small amount of serous fluid to lubricate the heart's movements and cushions it from any external jerk or shock.

Various solutions disclosed herein relate to devices and methods for placing anchors in the heart, particularly at the epicardium. Typically, anchors are embedded through the endocardium 20 and into the myocardium 21, which is the muscular, middle layer of the heart. For example, an anchor may be screwed into the myocardial tissue. In that situation, the strength of the anchor is dependent on the surface area of the screw that is dug into the tissue. However, the surface area of the screw may be fairly small in order to limit damage to the myocardium. Alternatively, by placing the anchor through the layers of the heart and past the epicardium 22, the entire surface area of one side of the anchor is in contact with the epicardium, creating a much stronger connection. A suture connected to the anchor in the epicardium 22 and going into a chamber of the heart can provide a strong attachment to any device tied to the anchor via the suture.

Such devices and methods can be used in medical procedures that implant a device in the heart permanently or temporarily (e.g., cinching mechanism during heart reshaping). In some examples, the anchors are implanted using a minimally invasive surgical procedure, such as a transfemoral approach through the femoral artery (large artery in the groin) or using a transapical approach with a small incision in the chest and entering through a large artery in the chest or through the tip of the left ventricle (the apex). Other approaches may also be used.

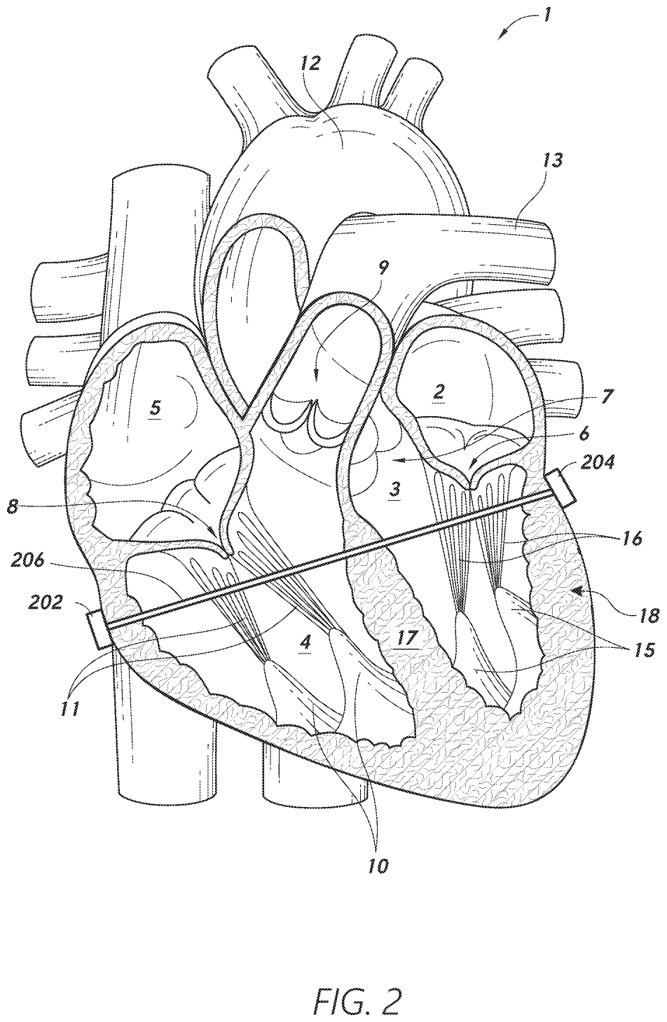

FIG. 2 illustrates one possible medical procedure where an anchor deployment device can be used. In some examples, anchors 202, 204 can be used to reshape the heart. A suture 206 is connected to the first anchor 202 and the second anchor 204. A cinching mechanism (not shown) can then be used to reel-in or otherwise shorten the suture 206, applying pressure on the heart walls at the anchors 202, 204. The cinching mechanism may be deployed between the two anchors or remain external to the body (e.g., an external winch connected to the suture through a catheter). The inward pressure pulls in the heart walls, reshaping the heart. For example, a more rounded heart can be squeezed to form a more normal, oblong shape.

The anchor deployment device may be utilized for either or both of the first anchor 202 and the second anchor 204. Optionally, different types of anchors may be used for the first and second anchors (e.g., any of the anchors described in this disclosure or other types of anchors). For example, some locations in the heart may have thinner walls, where smaller anchors may be desired. Variously shaped balloons (e.g., circular/disc-shaped/pie-shaped/cone-shaped configuration) may be deployed from the anchor deployment device and inflated into anchors. In one example, the deployed configuration of the anchor 202 (or other anchors described elsewhere herein) may provide a surface area of substantially 4 cm², which can effectively eliminate migration of the anchor into the tissue of the heart. Optionally, the surface area may be between 2 cm² and 6 cm² or between 3 cm² and 5 cm², though other sizes that are larger or smaller are also possible. Further, the anchor 202 may be configured to withstand forces due to tension within the suture 206 of up to substantially 17 Newtons (N). A larger surface area helps the anchor withstand higher forces. Further, the large surface area of the anchor and the centrally tension on the suture 206 cooperatively operate as a closure device which seals the punctures in the walls of the heart. The anchor may be formed of a material that allows tissue ingrowth into the material after implantation.

After initial deployment of the anchor 202 from a catheter, the suture 206 may be pulled. Tightening the suture (e.g., using a cinching mechanism) pulls the expandable anchor 202 against the epicardial surface of the heart wall, such that the anchor presses against the surface. The suture may be cords, wires, cables, etc. and may be rigid, semi-rigid, or flexible and may be elastic or non-elastic. An elastic suture may allow some give (e.g., expansion and contraction) during movement or beating of the heart, whereas a non-elastic suture may maintain the same or substantially the same relative distance between the superior and inferior anchors. The suture may optionally be braided or include a braided portion. The suture may be formed of a high strength/high performance polymers (e.g., ultra-high-molecular-weight polyethylene (UHMWPE), etc.).

While the above has discussed placing the anchor against the epicardium 22, some examples may place the anchor in a different location. For example, the anchor may be deployed past the epicardium 22, to one of the pericardium layers. In one example, rather than abutting against the epicardium, the anchor may be located externally to the pericardium and abut against the fibrous pericardium layer 25.

### Anchor Placement Device

As referenced above, certain examples disclosed herein provide for systems, devices and methods for placing an anchor for a medical device at the epicardium 22. Once anchored against the epicardium, such anchors can provide secure attachment to various types of medical implants or devices. The anchor placement device may be introduced into the patient system through surgical or, advantageously, minimally-invasive means.

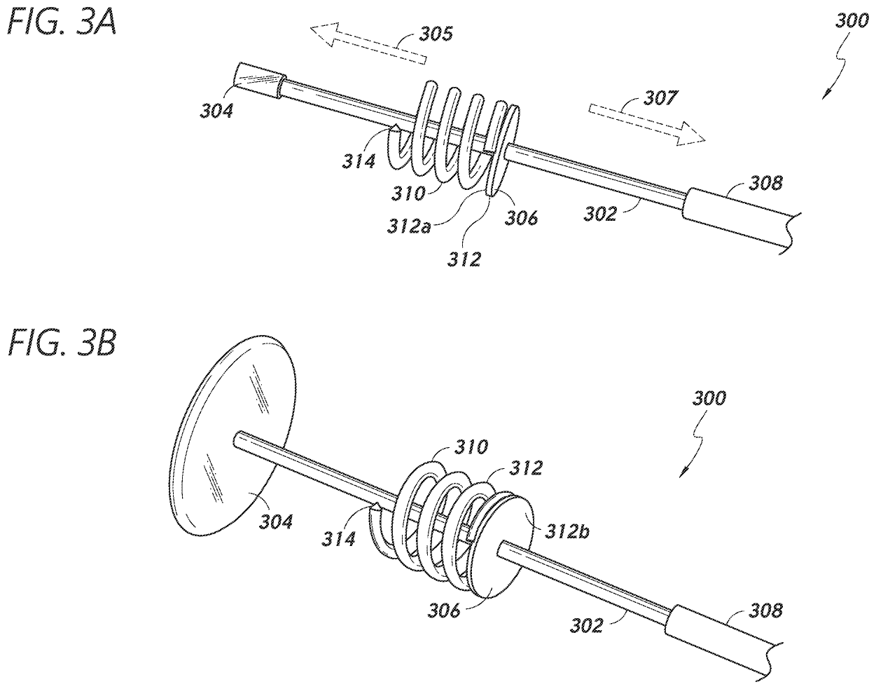

FIGS. 3A and 3B illustrate an example of the anchor placement device. The anchor placement device 300 comprises a tube 302 having an inflatable balloon 304 at a tip at the distal end. A needle (not shown) is formed at the distal end of the tube 302. A control plate 306 is located proximally from the balloon 304, along the tube 302. The tube 302 is delivered to a target site in the heart using a catheter 308.

For ease of explanation in FIGS. 3A-3B and other figures, components of the anchor placement device that are further away from the user are referred to as distal, along direction 305, while components that are closer to the user are referred to as proximal, along direction 307. For example, a handle of the catheter 308 operated by the user is described as at the proximal end, while the balloon 304 or the needle tip are described as at the distal end.

In some examples the tube 302 is a hollow structure with one or more lumens. For example, there may be a single lumen formed longitudinally by the walls of the tube or there may be interior wall(s) dividing the space within the tube into two or more separate lumens. In some examples, it may be desirable to keep certain components of the anchor placement device 300 separate from each other. The tube 302 and/or other components of the anchor placement device 300 may be formed of a biocompatible material, such as stainless steel, Nitinol, etc. In one example, the material is at least semi-rigid, to facilitate piercing the tube into the heart wall.

The lumen(s) of the tube 302 can be used to deliver components to the distal end of the tube 302. In one example, a curable adhesive or other liquid material that can be solidified is delivered from a reservoir attached to the proximal end of the tube 302, closest to an operator, through the lumen of the tube 302 and into the inflatable balloon 304. Filling the inflatable balloon 304 with the adhesive causes the balloon to expand into its anchor configuration, where the surface area available to abut against the epicardium 22 increases. With the larger surface area, the anchor can resist against more force.

In one example, an optical cable (not shown) is delivered to the target location through a lumen of the tube 302. The optical cable can be used to apply light (e.g., ultraviolet light) to the curable adhesive to solidify the adhesive. In some examples, the optical cable and the adhesive travel through the tube 302 in the same lumen. In other examples, the optical cable and adhesive travel through the tube in separate lumens, which may reduce interference by the adhesive with the optical cable as it moves through the tube 302.

In some examples, the control plate 306 comprises a helical spring structure 310 and a stopper plate 312. The spring structure 310 is distal from the stopper plate 312, with a proximal end of the spring structure 310 attached to the stopper plate 312. The tip 314 of the spring structure 310, at the spring's distal end, may be pointed to facilitate piercing heart tissue. In the illustrated example, the spring structure 310 coils around the tube 302 four times. Some examples may use more coils to more strongly attach the control plate 306 to heart tissue, with the additional coils allowing the spring structure 310 to be embedded more deeply into the tissue. Other examples may have fewer coils. The spring structure may be elastic or rigid. In some examples, the control plate 306 may use a different type of structure (e.g., a screw) to anchor itself into the heart wall.

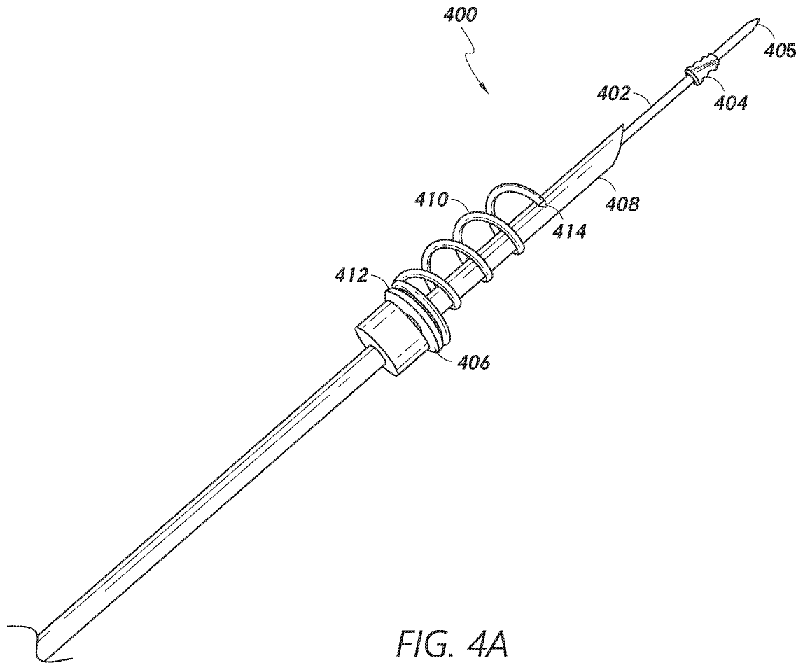

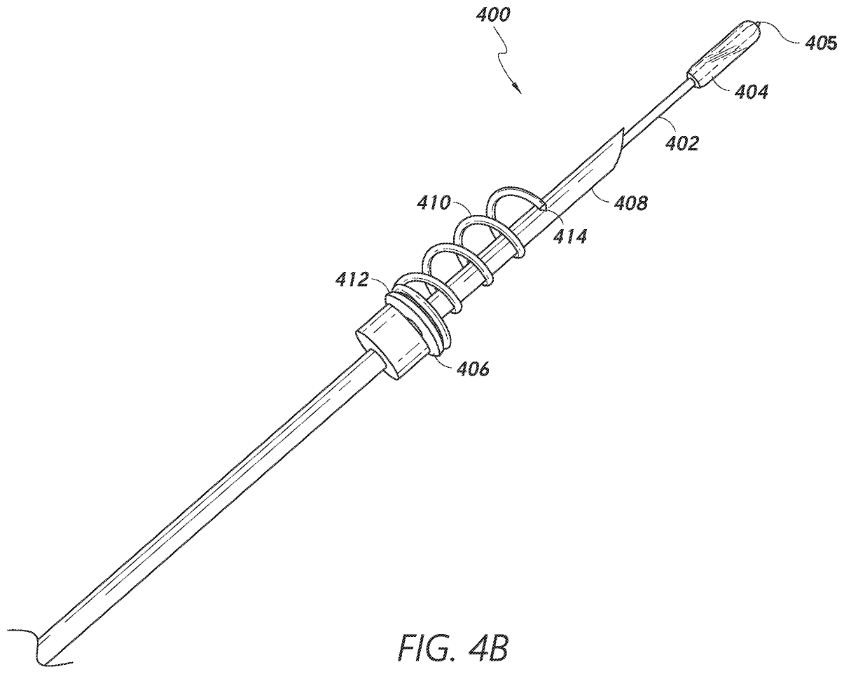

The control plate 306 may be attached at a fixed location on the tube 302, as shown in FIGS. 3A and 3B, or may be affixed to the catheter, as shown in FIGS. 4A and 4B. In some examples, the control plate 306 stabilizes the tube 302, to facilitate piercing the distal end of the tube 302 through the heart tissue and potentially minimize or reduce damage to the tissue. In some configurations, the control plate 306 functions by advancing the tip 314 of the spring to a surface of the endocardium 20 from the interior of the heart (e.g., in the left ventricle). The tube 302 can then be rotated to advance the tip 314 through the endocardium and into the myocardium 21. The control plate 306 can be advanced into the tissue until the surface of the endocardium abuts against the distal surface 312a of the stopper plate 312, opposite the proximal surface 312b of the stopper plate. The stopper plate 312 then prevents the control plate 306 from being advanced further into the tissue. In some examples, as the control plate 306 is advanced into the tissue, the tube 302 is also piercing into the tissue at the same time. If the control plate 306 is fixed to the tube 302, the control plate 306 prevents the end of the tube 302 from advancing further once the stopper plate 312 abuts against the endocardium 20. In this configuration, the control plate 306 provides depth control for how far the tube 302 advances through the heart walls. This may be beneficial in ensuring that the tube 302 advances past the epicardium 22, but not through further layers, such as the layers of the pericardium 24, 25.

In some examples, the control plate 306 may be movably fixed to the tube 302, allowing a user to set a desired piercing depth of the tube 302. For example, the control plate 306 may be releasably clamped to the tube 302, allowing the clamp to be loosened and the control plate 306 to be moved up or down on the tube. In some examples, depth indicators may be formed on the tube 302 to allow the user to select a desired depth.

In some examples, the control plate 306 may be collapsible to facilitate movement of the tube 302 through the catheter 308. In a first configuration, the control plate 306 can be collapsed to fit within the catheter 308. In a second configuration, as the control plate leaves the catheter, the control plate can expand back to its deployed state, in the form illustrated in FIG. 3A-3B. While the catheter is advanced to the target site with the anchor placement device 300 in the catheter, the control plate can be in the first configuration. As the anchor placement device is delivered to the target site and pushed out the end of the catheter, the control plate can be in the second configuration. In some examples, the control plate 306 may be made of a shape-memory alloy such as copper-aluminum-nickel and nickel-titanium (e.g., Nitinol) alloys and similar materials. In some examples, the control plate may utilize a mesh structure to facilitate collapsing to a smaller profile that can then expand when released from the catheter 308.

In FIG. 3A, the inflatable balloon 304 is shown in its unexpanded state. The balloon 304 is then inflated to put it into its anchor configuration, as shown in FIG. 3B, where its expanded state increases the surface area of side that will abut against the epicardium. For ease of explanation, the anchor configuration of the inflatable balloon will sometimes be referred to as an anchor 304. The anchor 304 may then be released from the tube 302 to be deployed at its target site. For example, the anchor 304 may be connected to the tube by a clamp or other releasable mechanism. In some examples, the inflatable balloon 304 includes an opening in the middle that enlarges when the balloon is inflated. For example, the balloon may be a toroidal shape when inflated. When inflated, the opening expands to allow the tube 302 to be pulled away from the balloon, allowing the balloon to be released. A suture (not shown) connected to the anchor allows the anchor to be connected to another medical device, such as an implant or another anchor.

FIGS. 4A and 4B illustrate another example of the anchor placement device. The example of the anchor placement device 400 has many similarities to the anchor placement device 300 of FIGS. 3A-3B, and many of the features described above in regard to that example also apply to anchor placement device 400 of FIGS. 4A-4B.

The anchor placement device 400 comprises a tube 402 having an inflatable balloon 404 at a tip at the distal end. A needle 405 is formed at the distal end of the tube 302. A control plate 406 is located proximally along the tube 302 from the balloon 304, closer to the user. The tube 402 is delivered to a target site in the heart using a catheter 308.

In comparison to the anchor placement device 300 example of FIGS. 3A-3B, the control plate 406 of the anchor placement device 400 is attached to the catheter 408. In some examples, the control plate 406 comprises a helical spring structure 410 and a stopper plate 412. The spring structure 410 is distal from the stopper plate 412, with a proximal end of the spring structure 410 attached to the stopper plate 412. The tip 414 of the spring 410, at the spring's distal end, may be pointed to facilitate piercing heart tissue.

In the illustrated example, the catheter 408 includes a slanted edge. In some examples, a slanted edge, a sharpened tip, a sharpened edge, or the like, formed at the distal end of the catheter 408 can aid in piercing tissue. In operation, the catheter 408 can pierce into the endocardium 20 from the interior of the heart (e.g., in the left ventricle). As the catheter 408 is advanced into the tissue, the tip 414 of the control plate 406 also begins piercing the tissue. The catheter 408, to which the control plate 406 is attached, may then be rotated to advance the tip 414 in a spiral path through the endocardium and into the myocardium 21. The control plate 406 can be advanced into the tissue until the surface of the endocardium abuts against the distal surface of the stopper plate 412 of the control plate 406. The stopper plate 412 then prevents the control plate 406, as well as the attached catheter 408, from being advanced further into the tissue.

With the control plate 406 anchored into the endocardium, the catheter 408 is kept from moving from the target site. The tube 402 can then be deployed from the catheter 408, through the heart tissue, until it reached past the epicardium 22. Once past the epicardium 22, the inflatable balloon 404 can be deployed from the tube 402, in similar fashion as described above with respect to FIGS. 3A-3B.

In FIG. 4A, the inflatable balloon 404 is shown in its unexpanded state. The balloon 404 is then inflated to put it into its anchor configuration, as shown in FIG. 4B, where its expanded state increases its surface area, particularly of the side of the balloon 404 that will abut against the epicardium. In the illustrated example, the balloon 404 expands to a cylindrical shape in proximity to the distal end of the tube 402. In FIG. 4B, the distal end of the expanded balloon 404 approaches, but does not reach the needle 405 tip. However, some examples may have an inflated balloon 404 that extends over the needle or is further back from the needle.

For ease of explanation, the anchor configuration of the inflatable balloon 404 is sometimes be referred to as an anchor 404. The anchor 404 may then be released from the tube 402 to be deployed at its target site. For example, the anchor 404 may be connected to the tube by a clamp or other releasable mechanism. In some examples, the inflatable balloon 404 includes an opening in the middle that enlarges when the balloon is inflated. For example, the balloon may be a toroidal shape when inflated. When inflated, the opening expands to allow the tube 402 to be pulled away from the balloon, allowing the balloon to be released. A suture (not shown) connected to the anchor allows the anchor to be connected to another medical device, such as an implant or another anchor.

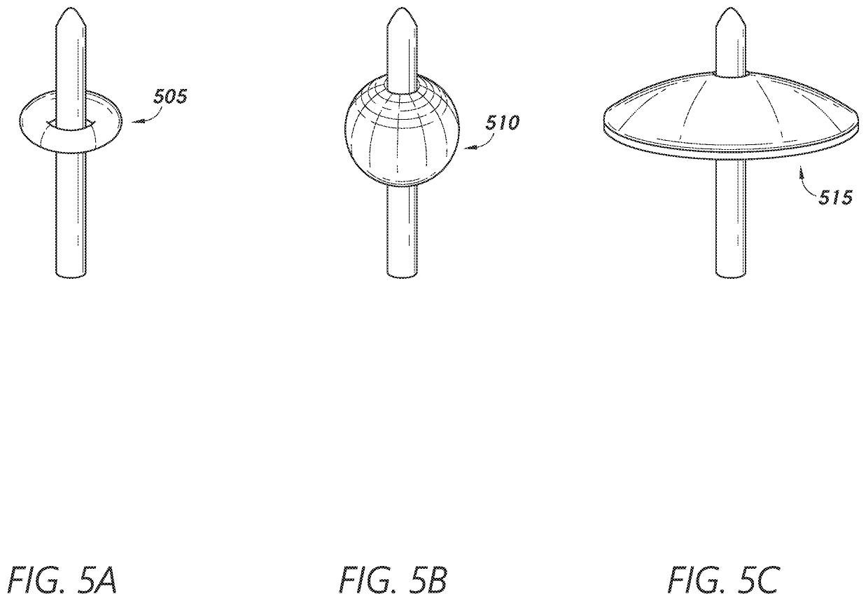

FIGS. 5A-5C illustrate some possible examples of the inflated shape of the balloon. For example, the balloon can a toroidal shape 505 (FIG. 5A), a spherical shape 510 (FIG. 5B), or a saucer shape 515 (FIG. 5C). Other examples of the balloon are also possible. Some shapes, with a greater surface area to abut against the epicardium 22 when the balloon is deployed as an anchor, may be able to provide greater resistance against pulling forces. For example, the saucer shape, like the disc shape of FIGS. 3A-3B, may provide stronger force resistance relative to other shapes due to the large surface area relative to the amount of material making up the balloon. These types of shapes have large distal and proximal surfaces, but have relatively short sides.

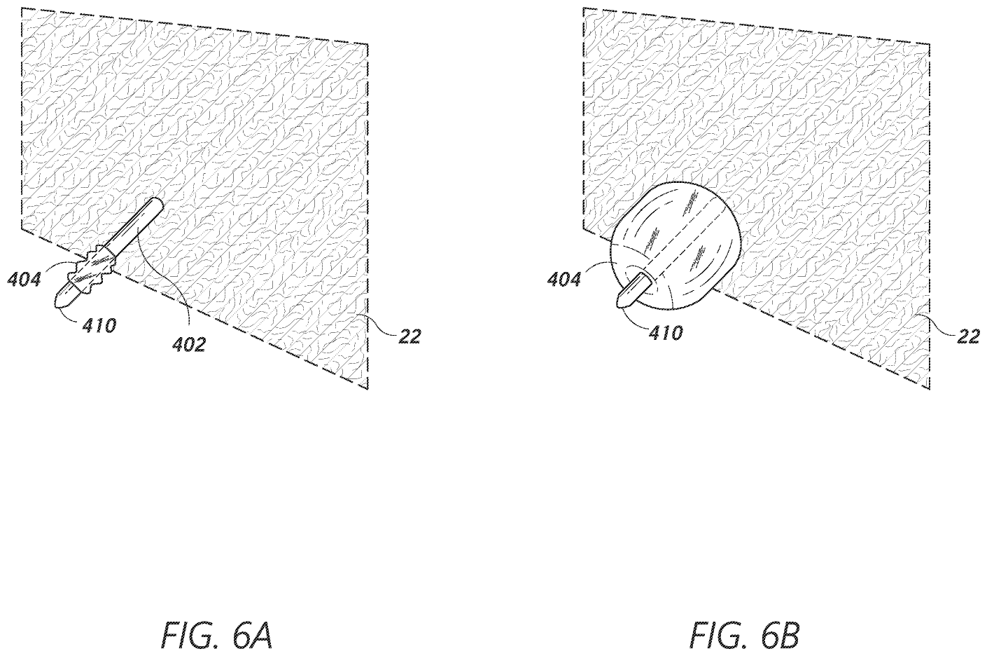

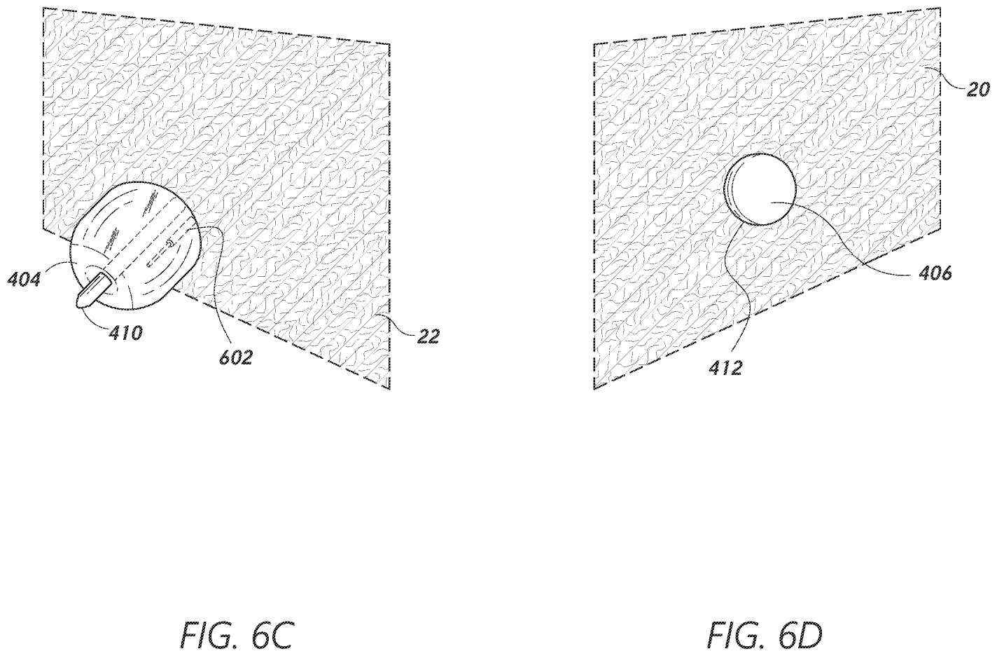

FIGS. 6A-6D illustrate a deployment of the inflatable balloon 404 into its anchor configuration, according to certain examples. For ease of explanation, FIGS. 6A-6D use the same labeling as FIGS. 4A-4B. However, the following can apply to other examples of the anchor placement device.

FIG. 6A illustrates a view of the side of the epicardium 22 facing away from the heart. As shown, the tube 402 of the anchor placement device 400 is breaking past the surface of the epicardium 22 from the interior of the heart. At the end of the tube 402 is a pointed end or needle 405 to aid in piercing the tissue. In near proximity to the needle 405 of the tube is a deflated balloon 404. For example, the balloon may be a few millimeters (e.g., 1 mm, 2 mm, etc. from the tip of the needle 405.

In FIG. 6B, the balloon 404 is inflated into its anchor configuration. In some examples, a curable adhesive is inserted into the balloon 404 to inflate it. By inflating the balloon 404, the surface area abutting the epicardium 22 increases, allowing the anchor to resist more inward force, towards the interior of the heart. For example, a suture may be connected to the anchor 404, which is then pulled inward to draw the walls of the heart closer together during a heart reshaping procedure.

FIG. 6C illustrates an optical cable 602 being deployed within the inflated balloon 404 to cure the adhesive or other settable material used to inflate the balloon 404. In some examples, the optical cable 602 transmits ultraviolet (UV) light to the adhesive to cure it. Once cured, the inflated balloon 404 is permanently set into its anchor configuration.

FIG. 6D illustrates a view of the side of the endocardium 20 facing the interior of the heart. Where FIG. 6B shows a view of the distal end of the anchor placement device 400, FIG. 6D shows a proximal view of the control plate 406. In FIG. 6D, the control plate 406 is inserted into the heart walls until the distal surface of the stopper plate 412 abuts against the endocardium 20.

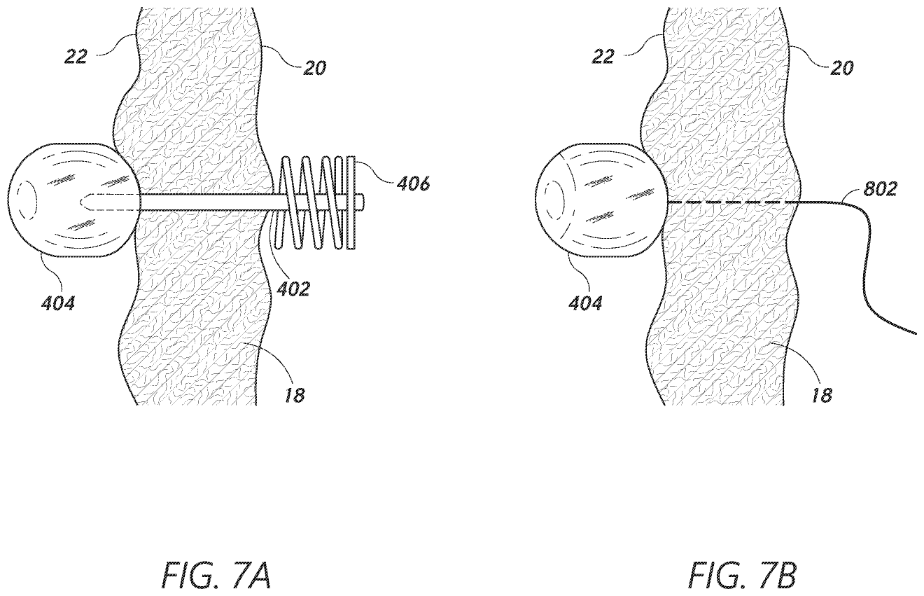

FIGS. 7A and 7B illustrate a side view of the anchor placement device 400 as the anchor 404 is detached from the tube 402, according to certain examples. In FIG. 7A, the tube 402 is shown withdrawing from the anchor 404. After the anchor 404 is released from its attachment point (e.g., a clamping mechanism, a compartment, etc.) on the tube 402, the tube 402 can be withdrawn from the anchor, while leaving the anchor in place.

In FIG. 7B, the tube 402 has been fully withdrawn, leaving the anchor 404 in place. A suture 802 is shown attached to the anchor. For example, the suture may be connected to the anchor via adhesive or tied to a hook, eyelet, or the like of the anchor 404. In one example, the suture may be embedded in the adhesive that is inserted into the balloon 404 to inflate it. When the adhesive is cured to set the balloon 404 into its anchor configuration, the suture also becomes firmly embedded in the cured adhesive, leaving the suture strongly attached to the anchor 404. In some examples, the cured adhesive serves as a secondary attachment mechanism for the suture, strengthening a primary (e.g., mechanical) attachment mechanism.

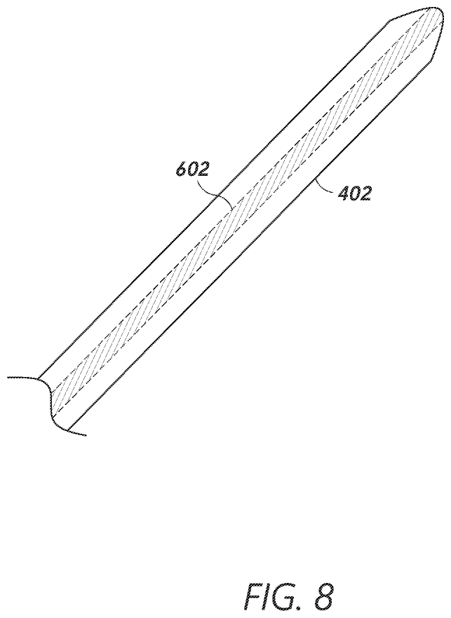

FIG. 8 illustrates a tube 402 of the anchor placement device 400 having an optical cable 602 built into its structure. The optical cable 602 may be formed on an exterior or interior surface of the tube. In one example, an optical material is built into a surface of the tube 402 and runs longitudinally along the tube. The optical material may be sheathed in cladding at least partly, with only a distal portion exposed where the balloon is located. For example, the optical material may be on an interior surface of the tube, and thus covered, for the majority of the tube's length, but then breaks through to the exterior surface where the balloon is located.

### Anchor Placement Device Usage

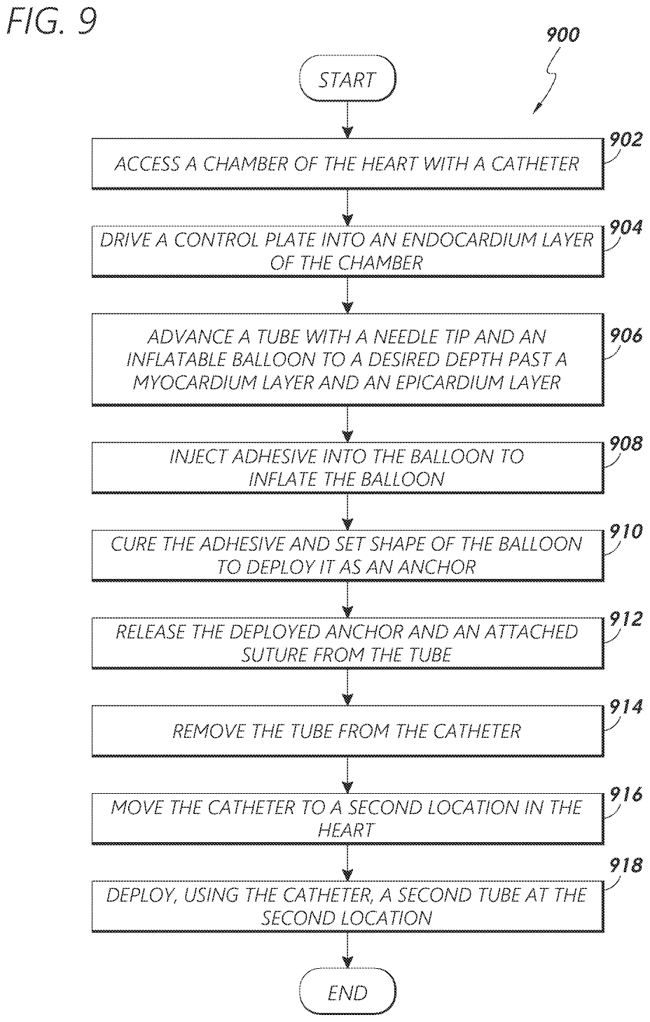

FIG. 9 provides a flow diagram representing a process 900 for using the anchor placement device 300, 400 to perform a medical procedure, according to one or more examples disclosed herein. A health provider, such as a surgeon, can use the process during a transseptal, trans aorta, transfemoral artery, trans radial, transapical, or other surgical approach to perform a medical procedure (e.g., installing a stent, valve, or other implant). In addition, for ease of explanation, the following uses the label numbering from FIGS. 4A-4B. However, the process is not limited to the specific example illustrated in those figures. For example, the example of FIG. 3A-3B, as well as other examples of the anchor placement device can perform the process 900.

In addition, the following process 900 describes deploying the anchor 404 past the epicardium layer 22 of the heart and deploying from the left ventricle. However, different examples of the anchor may be deployed in different layers of the heart or within different chambers of the heart. For example, some anchors may be deployed past the serious pericardium 24 or the fibrous pericardium 25.

At block 902, the anchor placement device 400 accesses a chamber of the heart, such as a left ventricle, with a catheter 408. Any of a variety of approaches can be used to reach the chamber with the catheter 408, such as a transeptal or transfemoral approach. Preferably, a minimally invasive procedure is used to reduce damage and shorten recovery times. The anchor placement device 400 is introduced into the chamber through the catheter 408.

At block 904, a control plate 406 of the anchor placement device 400 is driven into an endocardium layer 20 of the chamber. The control plate 406 may also dig into a myocardium layer 21 of the chamber to create a stronger attachment. Various examples of the control plate can be used to stabilize the anchor placement device 400. In some examples, the control plate can comprise of one or more needles, screws, adhesive, or the like for attaching the anchor placement device 400 to the endocardium. In one example, the control plate 406 comprises a helical spring structure 410 for digging into the walls of the chamber, as discussed in FIGS. 3A-4B. In that example, the control plate 406 is rotated (e.g., clockwise) to dig the spring structure into the heart walls.

At block 906, a tube 402 of the anchor placement device 400 with a needle 405 and an inflatable balloon 404 is advanced to a desired depth through the heart wall, past the myocardium layer 21 and through an epicardium layer 22. In an example operation, only the distal portion of the tube, comprising the needle and the inflatable balloon 404 (in an uninflated state), breaks past the epicardium layer 22, while the remaining portion of the tube 402 is inside the epicardium layer 22, the myocardium layer 21, and the endocardium layer 20. In some examples, the catheter 408 may be partially inserted into the heart wall, sheathing at least a portion of the tube 402.

At block 908, adhesive or another settable liquid is injected into the balloon 404 to inflate the balloon into its anchor configuration. The balloon 404 may be located in the pericardial cavity 23, between the epicardium 22 and the serous pericardium 24, providing a space for the balloon to be inflated without or with only limited resistance from the surrounding tissue.

As discussed previously, the balloon's anchor configuration can be one of various different shapes, such as a generally saucer shape, a generally cylindrical shape, a generally spherical shape, a generally disc shape, or the like. In general, shapes with a larger surface area that can abut against the epicardium layer 22 will provide a stronger resistance to pulling forces acting on the anchor. However, smaller shapes may be useful if the desired force resistance is relatively low and a smaller footprint is desired (e.g., to minimize damage and aid in recovery).

At block 910, the adhesive is cured, setting the shape of the balloon 404 permanently to deploy it as an anchor. In some examples, an optical cable 602 is deployed into the balloon 404 and UV light emitted from the cable in order to cure the adhesive. In some examples, the adhesive may cure on its own with time. In another example, a second compound may be added to the adhesive to promote curing, such as in two-component adhesives that use a resin and a hardener.

At block 912, the deployed anchor 404, along with a suture or other connector, is released from the tube 402. Various types of release mechanisms can be used to release the anchor 404 from the tube 402. For example, there may be a clamping mechanism that holds the balloon against the tube. In another example, the balloon 404 may be attached to the tube via a friction fit, where the undeployed balloon is constricted against the tube, keeping it in place. As the balloon 404 is expanded the friction fit is released, allowing the balloon to be separated from the tube 402. In one example, the balloon 404 may fit into a compartment, groove, or other space in the tube 402. As the balloon 404 is expanded, the balloon 404 leaves the space, releasing it from the tube 402.

At block 914, the tube 402 is removed from the catheter. As the anchor 404 has been deployed from the catheter, a new tube 402 with a second deployable balloon 404 needs to be inserted in the catheter to deploy another anchor. In some examples, the tube 402 may have multiple balloons, allowing the same tube to deploy multiple anchors. However, the following steps assume that each tube 402 includes a single balloon 404.

At block 916, the catheter is moved to a second location in the heart. The second location is another site in the heart where an anchor needs to be deployed. For example, a heart reshaping procedure may require installing a first anchor 202 and a second anchor 204, as shown in FIG. 2. However, in some medical procedures, moving to the second location in unnecessary. For example, an implant being attached to the deployed anchor 404 may only need a single anchor to keep it in place, thus a installing a second anchor is not needed.

At block 918, a second tube is deployed at the second location, using the catheter. The second anchor can then be deployed from the second tube. While the above has discussed deploying two anchors, any number of anchors can be deployed. For example, if only a single anchor is needed, the process 900 can end after block 912 by removing the anchor deployment device from the heart. Some medical procedures may require installing three, four, or more anchors in the heart. In those situations, the catheter can be moved to a third location, a fourth location, etc., to deploy additional anchors.

### Additional Description of Examples

Provided below is a list of examples, each of which may include aspects of any of the other examples disclosed herein. Furthermore, aspects of any example described above may be implemented in any of the numbered examples provided below.

Example 1: A device for placing an anchor in a heart, the device comprising: a tube; a needle formed on a distal end of the tube, the needle configured to pierce from an endocardium layer of the heart through an epicardium layer of the heart; an inflatable balloon in proximity to the distal end of the tube, the inflatable balloon configured to inflate and form an anchor located outside the epicardium layer; and a control plate located proximally from the inflatable balloon, the control plate configured to abut against the endocardium layer and reduce movement of the tube while the inflatable balloon is inflated.

Example 2: The device of any example herein, in particular example 1, further comprising: a conduit in the tube for releasing curable adhesive into the inflatable balloon, the curable adhesive configured to inflate the balloon.

Example 3. The device of example 2, further comprising: a fiber optic cable configured to emit ultraviolet (UV) light, the UV light configured to cure the adhesive in the inflatable balloon and permanently leave the inflatable balloon in its inflated configuration.

Example 4. The device of example 3, wherein the fiber optic cable runs longitudinally along a surface of the tube.

Example 5. The device of example 3, wherein the fiber optic cable and the conduit are in separate lumens in the tube.

Example 6. The device of any one of examples 1 to 5, further comprising: a catheter configured to contain the tube; wherein the control plate is attached to an external surface of the catheter, the control plate configured to hold the catheter steady while the tube and inflatable balloon are moved to a target location.

Example 7. The device of any of examples 1 to 5, wherein the control plate is attached to the tube at a fixed distance from the needle, the control plate configured to prevent the needle from passing further than the fixed distance from the endocardium layer.

Example 8. The device of any of examples 1 to 7, wherein the inflatable balloon is configured to form a generally disk shape.

Example 9. The device of any of examples 1 to 7, wherein the inflatable balloon is configured to form a generally cylindrical shape.

Example 10. The device of any of examples 1 to 7, wherein the inflatable balloon is configured to form a generally saucer shape.

Example 11. The device of any of examples 1 to 10, further comprising a suture attached to the inflatable balloon.

Example 12. The device of any of examples 1 to 11, wherein the inflatable balloon is further configured to detach from the tube.

Example 13. A method for deploying an anchor to an epicardium layer of a heart, the method comprising: advancing a tube to an interior chamber of the heart, the tube comprising a needle formed on a distal end of the tube, an inflatable balloon in proximity to the distal end of the tube, and a control plate located proximally from the inflatable balloon; anchoring the control plate to an interior surface of an endocardium layer of the heart; advancing the needle from the endocardium layer of the heart through an epicardium layer of the heart; and deploying the inflatable balloon to form an anchor against an exterior surface of the epicardium layer.

Example 14. The method of example 13, wherein deploying the inflatable balloon comprises: releasing a curable adhesive into the inflatable balloon to expand the inflatable balloon to a target size; and curing the adhesive within the inflatable balloon to permanently set the inflatable balloon into an anchor configuration.

Example 15. The method of example 14, wherein curing the adhesive comprising: deploying an ultra-violet (UV) emitting cable into the inflatable balloon to apply UV light to the curable adhesive; and setting the adhesive into a rigid material that forms the inflatable balloon into the anchor configuration.

Example 16. The method of any of examples 13 to 15, further comprising: releasing the inflatable balloon from the tube, leaving the inflatable balloon anchored to the epicardium layer, the inflatable balloon attached to a suture; and withdrawing the tube from the interior chamber of the heart.

Example 17. The method of any of examples 13 to 16, further comprising: advancing a catheter containing the tube to the interior chamber of the heart, the control plate attached to an exterior surface of the catheter.

Example 18. The method of example 17, further comprising: releasing the inflatable balloon from the tube, leaving the inflatable balloon as a first anchor at the epicardium layer, the inflatable balloon attached to a suture; withdrawing the tube from the catheter; moving the catheter to a second location; advancing a second tube having a second inflatable balloon into the catheter; and deploying the second inflatable balloon as a second anchor at the second location.

Example 19. The method of example 18, further comprising: deploying a cinching device connected, via a suture, to the first anchor and the second anchor; and cinching the suture to reduce a size of a structure of the heart, the structure located between the first anchor and the second anchor.

Example 20. A needle for placing an anchor in a heart, the needle configured to pierce from an endocardium layer of the heart through an epicardium layer of the heart, the needle comprising: an inflatable balloon in proximity to a distal end of the needle, the inflatable balloon configured to: inflate and form an anchor located outside the epicardium layer; and detach from the needle; a suture attached to the inflatable balloon; and a control plate located proximally from the inflatable balloon, the control plate configured to abut against the endocardium layer and reduce movement of the needle while the inflatable balloon is inflated.

Depending on the example, certain acts, events, or functions of any of the processes described herein can be performed in a different sequence, may be added, merged, or left out altogether. Thus, in certain examples, not all described acts or events are necessary for the practice of the processes. Moreover, in certain examples, acts or events may be performed concurrently, rather than sequentially.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be understood that certain ordinal terms (e.g., “first” or “second”, “primary” or “secondary”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” “proximal,” “distal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

It should be appreciated that in the above description of examples, various features are sometimes grouped together in a single example, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

What is claimed is:

1. A device for placing an anchor in a heart, the device comprising:
a tube;
a needle formed on a distal end of the tube, the needle configured to pierce from an endocardium layer of the heart through an epicardium layer of the heart;
an inflatable balloon in proximity to the distal end of the tube, the inflatable balloon configured to inflate and form an anchor located outside the epicardium layer; and
a control plate located proximally from the inflatable balloon, the control plate configured to abut against the endocardium layer and reduce movement of the tube while the inflatable balloon is inflated.

2. The device of claim 1, further comprising:
a conduit in the tube for releasing curable adhesive into the inflatable balloon, the curable adhesive configured to inflate the inflatable balloon.

3. The device of claim 2, further comprising:
a fiber optic cable configured to emit ultraviolet (UV) light, the UV light configured to cure the curable adhesive in the inflatable balloon and permanently leave the inflatable balloon in its inflated configuration.

4. The device of claim 3, wherein the fiber optic cable runs longitudinally along a surface of the tube.

5. The device of claim 3, wherein the fiber optic cable and the conduit are in separate lumens in the tube.

6. The device of claim 1, further comprising:
a catheter configured to contain the tube;
wherein the control plate is attached to an external surface of the catheter, the control plate configured to hold the catheter steady while the tube and inflatable balloon are moved to a target location.

7. The device of claim 1, wherein the control plate is attached to the tube at a fixed distance from the needle, the control plate configured to prevent the needle from passing further than the fixed distance from the endocardium layer.

8. The device of claim 1, wherein the inflatable balloon is configured to form a generally disk shape.

9. The device of claim 1, wherein the inflatable balloon is configured to form a generally cylindrical shape.

10. The device of claim 1, wherein the inflatable balloon is configured to form a generally saucer shape.

11. The device of claim 1, further comprising a suture attached to the inflatable balloon.

12. The device of claim 1, wherein the inflatable balloon is further configured to detach from the tube.

13. A method for deploying an anchor to an epicardium layer of a heart, the method comprising:
advancing a tube to an interior chamber of the heart, the tube comprising a needle formed on a distal end of the tube, an inflatable balloon in proximity to the distal end of the tube, and a control plate located proximally from the inflatable balloon;
anchoring the control plate to an interior surface of an endocardium layer of the heart;
advancing the needle from the endocardium layer of the heart through an epicardium layer of the heart; and
deploying the inflatable balloon to form an anchor against an exterior surface of the epicardium layer.

14. The method of claim 13, wherein deploying the inflatable balloon comprises:
releasing a curable adhesive into the inflatable balloon to expand the inflatable balloon to a target size; and
curing the curable adhesive within the inflatable balloon to permanently set the inflatable balloon into an anchor configuration.

15. The method of claim 14, wherein curing the curable adhesive comprises:
deploying an ultra-violet (UV) emitting cable into the inflatable balloon to apply UV light to the curable adhesive; and
setting the curable adhesive into a rigid material that forms the inflatable balloon into the anchor configuration.

16. The method of claim 13, further comprising:
releasing the inflatable balloon from the tube, leaving the inflatable balloon anchored to the epicardium layer, the inflatable balloon attached to a suture; and
withdrawing the tube from the interior chamber of the heart.

17. The method of claim 13, further comprising:
advancing a catheter containing the tube to the interior chamber of the heart, the control plate attached to an exterior surface of the catheter.

18. The method of claim 17, further comprising:
releasing the inflatable balloon from the tube, leaving the inflatable balloon as a first anchor at the epicardium layer, the inflatable balloon attached to a suture;
withdrawing the tube from the catheter;
moving the catheter to a second location;
advancing a second tube having a second inflatable balloon into the catheter; and
deploying the second inflatable balloon as a second anchor at the second location.

19. The method of claim 18, further comprising:
deploying a cinching device connected, via a suture, to the first anchor and the second anchor; and
cinching the suture to reduce a size of a structure of the heart, the structure located between the first anchor and the second anchor.

20. A needle for placing an anchor in a heart, the needle configured to pierce from an endocardium layer of the heart through an epicardium layer of the heart, the needle comprising:
an inflatable balloon in proximity to a distal end of the needle, the inflatable balloon configured to:
inflate and form an anchor located outside the epicardium layer; and
detach from the needle;
a suture attached to the inflatable balloon; and
a control plate located proximally from the inflatable balloon, the control plate configured to abut against the endocardium layer and reduce movement of the needle while the inflatable balloon is inflated.
